(19) United States
(12) Patent Application Publication
(10) Pub. No.: US 2025/0118177 A1
(43) Pub. Date: Apr. 10, 2025

(54) LATERAL AWARENESS ASSISTANCE SYSTEM

(71) Applicant: OCUTECH, INC., Chapel Hill, NC (US)

(72) Inventors: Henry A. GREENE, Durham, NC (US); Dominic GERMANA, Durham, NC (US); Brad FORREST, Cary, NC (US)

(73) Assignee: OCUTECH, INC., Chapel Hill, NC (US)

(21) Appl. No.: 18/908,441

(22) Filed: Oct. 7, 2024

Related U.S. Application Data
(60) Provisional application No. 63/543,150, filed on Oct. 9, 2023.

(51) Int. Cl. G08B 6/00; G02C 11/00; G08B 5/36; G16H 40/67

(52) U.S. Cl. CPC G08B 6/00; G02C 11/10; G16H 40/67; G02C 2200/02; G08B 5/36

(57) ABSTRACT

A lateral awareness system, which may be configured to be worn by a user using glasses for example, may include: a transducer, a power source to power the transducer, a processor and an alerting system. The transducer is configured to only monitor whether objects are within one lateral side zone of the user. The processor is configured to receive and process signals from the transducer to determine when alerts should be generated. The alerting system is configured to generate the alerts when an object is in the lateral side zone of the user.

### BACKGROUND

It is estimated that there are between 500,000 and 1 million individuals in the US who have a one-sided loss of their vision, called Homonymous Hemianopia (HH) (also called Homonymous Hemianopia). While HH is most frequently a result of stroke, it can also come from a range of other causes amongst them head trauma, brain tumors and developmental disorders. HH is not the same as loss of vision in one eye-individuals with no vision in one eye can still see to both sides of their line of sight with their remaining eye. In Homonymous Hemianopia, on the other hand, depending upon which half of the brain is damaged, neither eye will have vision to the affected side. If the right brain is involved, the vision loss is on the left side, and if the left brain is affected the right visual field will be lost. HH can have a much more profound impact on visual functioning, mobility and independence than does loss of one eye.

Studies have demonstrated that people with hemianopic visual field defects have an increased risk of accidents. For example, people report walking into objects, tripping and falling, feeling unsafe, getting lost, and experiencing panic when in crowded or unfamiliar areas (Robinson et al.). Studies have also found that patients with HH were deeply concerned about their ability to move independently. The homonymous hemianopic visual field defect interfered with patients' daily living activities related to movement.

### SUMMARY

Aspects to at least some embodiments of the present application relate to a device attachable to conventional eyeglass frames that will notify the wearer who has a visual field loss to the right, left or to both sides of their visual field of a potential obstacle which they may encounter.

In some embodiments, an ultrasonic transceiver will cause a haptic to vibrate, tap, etc. to notify the individual of the potential obstacle in their non-seeing side of their visual field. Firmware may be employed to allow the device to respond to obstacles at varying distances and degrees of eccentricity both vertically and horizontally with a range of notification signals.

According to some embodiments, the sensor is configured to be attached to eyeglass frame temples of varying sizes as well as to be aimed at different vertical angles to accommodate differences in the angle of the eyeglass frame temples as well as to vary the angular field of view of the sensor up and down to best address the individual user's posture and mobility needs.

An advantage of some aspects of the disclosure is to solve at least a part of the problems described above, and aspects of the disclosure can be implemented as the following aspects.

One aspect of the disclosure is a lateral awareness system configured to be worn by a user. The lateral awareness system may include: a transducer, a power source to power the transducer, a processor and an alerting system. The transducer is configured to only monitor whether objects are in a lateral side zone of a user, and the processor is configured to receive and process signals from the transducer to determine when alerts should be generated. The alerting system is configured to generate the alerts when an object is in the lateral side zone at a distance and position that may cause a collision with the user.

The skilled person will appreciate that except where mutually exclusive, a feature described in relation to any one of the above embodiments may be applied mutatis mutandis to any other embodiment.

### BRIEF DESCRIPTION OF THE DRAWINGS

The disclosure will be described with reference to the accompanying drawings, wherein like numbers reference like elements.

FIGS. 1A and 1B are schematics of a front view of a person and top view of the head of that person, respectively.

FIG. 2 is a top view of glasses with a lateral awareness system according to some embodiments.

FIG. 3A is a perspective view of glasses with a lateral awareness system according to some embodiments.

FIG. 3B is a perspective view of the glasses with an exploded view of the lateral awareness system of FIG. 3A according to some embodiments.

FIGS. 4A, 4B and 4C are perspective views of the lateral awareness system including a fastening or attachment system, according to some embodiments.

FIGS. 5 and 6 are schematic diagrams of a lateral awareness system, according to some embodiments.

FIG. 7 is a top view of glasses with a lateral awareness system and multiple detection zones, according to some embodiments.

FIG. 8 is method of operating a lateral awareness system with multiple detection zones, according to some embodiments.

### DESCRIPTION OF EXEMPLARY EMBODIMENTS

As mentioned briefly above, a common visual effect of brain injury or stroke is the loss of the person's visual field or our ability to see to the side. There are many types of visual field losses that can occur, but the most common form is HH or loss of half of the field of vision in each eye. If the posterior portion of the brain is damaged on one side of the brain, a loss of visual field occurs to the opposite side in both eyes. Patients often mistakenly believe the loss is just in one eye. When certain portions of the brain are damaged, the patient may also fail to appreciate space to one side, which is usually to the left. Unlike visual field loss, this problem is not a physical loss of sensation, but rather a loss of attention to the area. Unilateral neglect is a disorder of attention where patients are unable to attend to stimuli, such as objects and people, located on one side of space. It most commonly results from brain injury or stroke to the right cerebral hemisphere, causing visual neglect of the left-hand side of space.

This disclosure relates to a system to alleviate disadvantages for persons having such disorder but should not be limited to persons experiencing HH and can be applied to any application where there is a desire to become aware of objects proximate to a person, device/machine or item. Thus, while this invention is not limited to applications for HH, embodiments of the invention are described below using applications related to HH.

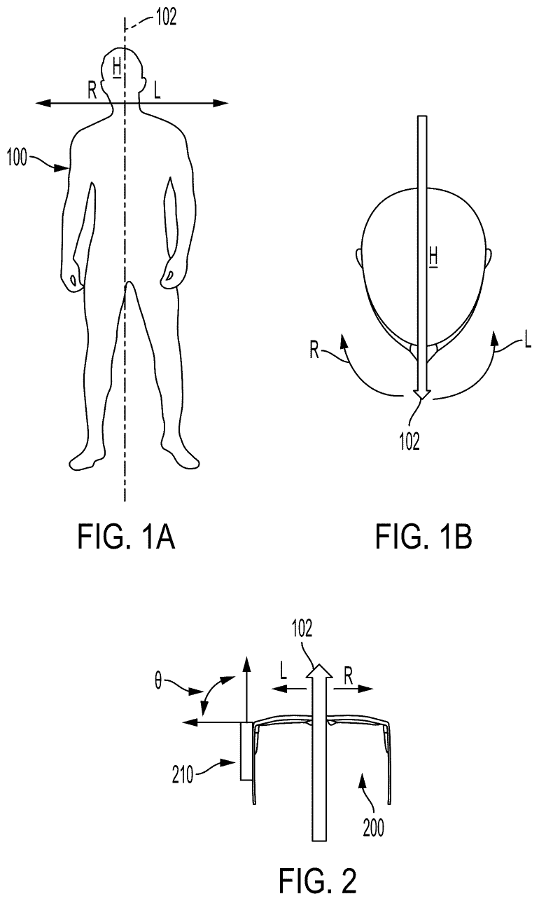

FIG. 1A illustrates a schematic front view of a person 100 and FIG. 1B shows a schematic top view of a head H of the person 100. The arrow R in the right hemisphere of the person 100 represents an unimpaired field of view, whereas the person's vision in his left hemisphere, indicated by arrow L, is impaired, for example due to visual neglect or visual field loss. The area indicated in FIGS. 1A and 1B show the user's line of sight 102 that is straight in front of the eyes of the person 100.

The lateral awareness assistance system 210 according to some embodiments may be applied to glasses 200 attached to the person's head H. Examples of such glasses 200 are shown in FIG. 2 such that the assistance system 210 may be attached or mounted. The lateral awareness assistance system 210 is configured to detect objects to only one side of the user's line of sight 102, such as, for example in FIG. 2, only the area denoted by an L or only the lefthand side of the user's line of sight 102 which can compensate for vision loss in such area.

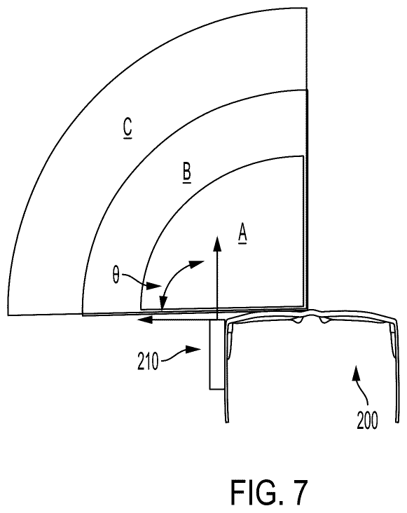

In addition, in one embodiment, the lateral awareness assistance system 210 is also configured to detect objects only in front of the user 100, which is shown in an example in FIG. 7 (which is discussed in more depth later herein). Thus, in one embodiment, the lateral awareness assistance system 210 is configured to only detect objects to one side of the user's line of sight 102 and only in front of the user. This allows a user that is walking that has impaired vision on one side of the user's body to detect objects that will likely be in the user's walking path (or at least close to such walking path).

As shown in FIG. 2, the lateral awareness assistance system 210 is configured to only detect objects within a certain angle θ, which could be any angle, such as 45 degree section, 60 degree section, 90 degree section, 135 degree section, 180 degree section, etc.). In one embodiment, the system 210 employs the detection zone as detecting objections within a θ of 45 degrees to the non-seeing side from the central line of sight 102 of area that is outside the user's line of sight area 102.

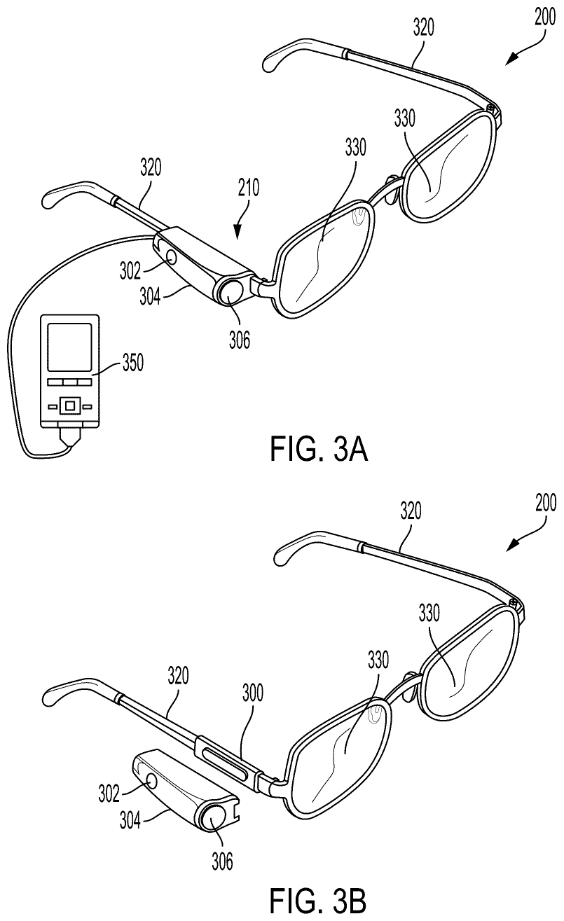

FIGS. 3-6 illustrate components of the assistance system 210 which are configured to be attached to one or more temples 320 of a pair of glasses 200. It should be noted that the eyeglasses 200 include lenses 330 for the user to look through but are not required.

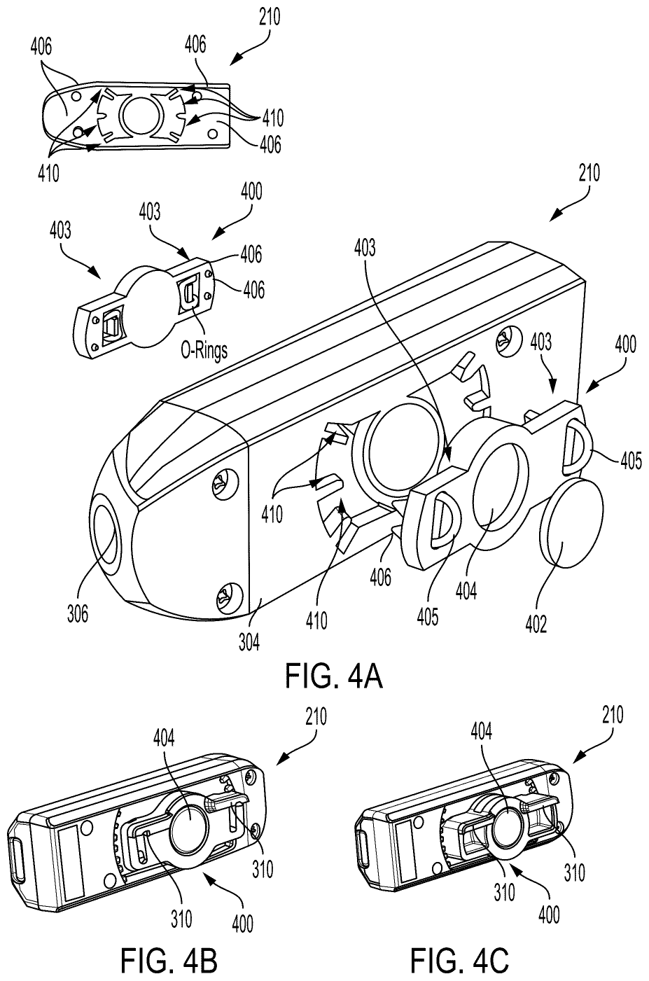

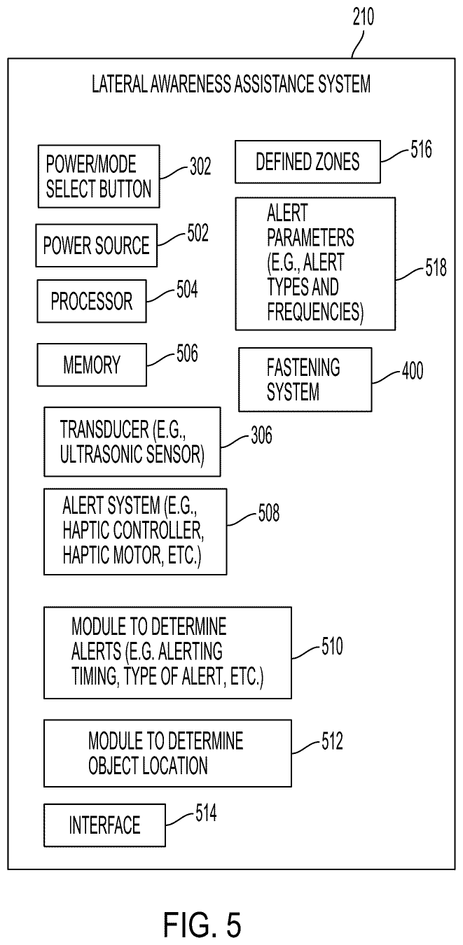

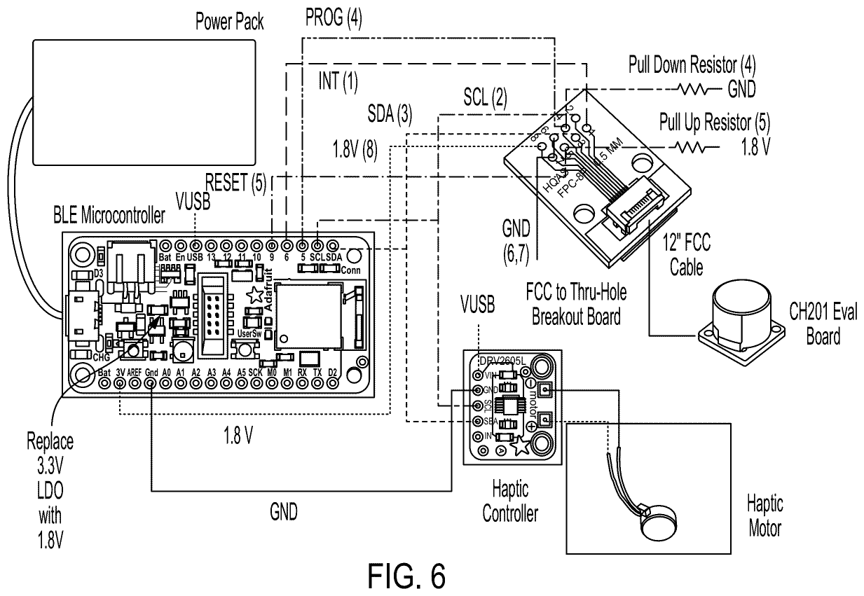

The assistance system 210, as shown in FIGS. 3-6, may include a housing 304, a transducer (e.g., an ultrasonic sensor 306), a fastening system 300 and a power/mode-select button 302. The housing 304 shown in FIGS. 4A-4C house the electronic components, which includes a printed circuit board, processor 504, memory 506, transducer 306, an alert system 508 (haptic), battery, and the like. Other components of the assistance system 210 may include a power source 502, modules 510, 512, interface(s) 514, and data stored in memory including defined zones 516, and alert parameters 518. The components shown in FIGS. 5 and 6 will be discussed later herein.

Referring back to FIGS. 3A-3B, the housing 304 may be a horn design that directs the receptive field of the sensor that is molded into the housing 304, according to an embodiment. The horn design is wrapped around the sensor so that the sensor only sends signals in a direction that is not omnidirectional but instead is directional about an angle θ (e.g., 45 degrees in front of the user and to one side of the user's line of sight 102 as shown in FIG. 2).

Alternatively, or in addition, the design of the housing 304, according to an embodiment, may provide a sensor position that is not parallel to the line of sight of the user so that the sensing field is directed from the central line of sight laterally and vertically into the non-seeing lateral field of view.

While FIGS. 3A-3B show the system is only on one of the temples 320 of the glasses 200, it should be understood that the system 210 may be on both temples 320 of the glasses 200 to monitor both the right and left lateral field of the user's vision.

FIGS. 4A, 4B and 4C are perspective views of the lateral awareness system including a fastening or attachment system, according to some embodiments.

In FIG. 4A, the fastening system 400 includes two wings 403, a self-adhesing rubber disk 402, a circular magnet 404 in between the two wings 403, angled pins 406 on each wing 403, and an o-ring 405 at each wing 403. The housing 304 of the lateral awareness assistance system 210 includes a recessed area on one side which has ribs 410. The angled pins 406 capture each side of the ribs 410 to connect the fastening system 400 to the housing 304 of the lateral awareness assistance system 210. The o-rings and the magnet 404 are then configured to connect to the temples 320 of glasses 200 to the lateral awareness assistance system 210. In other embodiments of FIGS. 4B-4C, the system is similar to FIG. 4A except, instead of o-rings, a clip (not shown), inserted into the mounting apparatus, will allow it to be attached to the metal temples 320 of glasses while a ridge 310 of the fastening system rests on the glasses temple 320 so that the lateral awareness assistance system 210 is fully supported in a vertical direction.

Also, in FIG. 4C, a clip is inserted into the mounting apparatus so that the device can be clipped on and off the eyeglass frame temples. The magnet allows the device itself to be attached to the fastening system. The ‘teeth’ on the fastening system and on the inside of the device itself allow for the device to be angled to direct the sensor in the proper direction.

In each of the systems shown in FIGS. 4A-4C, the fastening system 400 includes a circular protrusion that inserts into a circular recess of the housing 304. This allows for the housing 304 of the lateral awareness assistance system 210 to rotate in an axis perpendicular to the line of sight of the user relative to the fastening system (and thus, relative to the glasses). When desired, the user can lock a desired position of the fastening system 400 relative to the housing.

Referring now to FIGS. 5-6, FIGS. 5 and 6 are schematic diagrams of a lateral awareness system, according to some embodiments. The components, which were briefly introduced above, are explained in detail below, according to some embodiments.

The processor 504 (also referred to herein as the “controller” or “microcontroller”) may be a CPU and is configured to execute instructions and perform one or more of the tasks/steps discussed herein. The processor 504 may be more than one processor and is in communication with memory 506 and one or more other components listed in FIG. 6. The processor 504 also is configured to control one or more of the components shown in FIG. 5, such as the alerting system 508, and ultrasonic sensor 306. For example, the processor 504 is configured to control the alerting system 508 to output an alert to the user when a signal is received from the ultrasonic sensor 306 that is greater than a preset threshold.

The power source 502 is a battery. The power source 502 is configured to supply electric power to the electronic components of the system, including the processor 504 and the ultrasonic sensor 306.

The memory 506 includes a ROM and a RAM and various computer programs are stored in the ROM. The processor 504 develops or loads, in the RAM, the computer programs or modules stored in the ROM to execute the computer programs or modules. The computer programs/modules 510, 512 include computer programs for determining an object entering, being in, or exiting one or more detection areas, as explained below.

The interface 514 is an input/output interface for connecting various external devices, which are supply sources of contents, to the modules 510, 512. Examples of the external devices include a personal computer (PC), a cellular phone terminal, and a controller device 350. As the interface, for example, a USB interface, a micro USB interface, and an interface for a memory card can be used.

The computer programs or modules include computer readable instructions for determining if an object is within a predetermined zone and if so, providing a corresponding alert using the alerting system 508 as explained below.

The alerting system 508 may include any alerting system to alert any of a person's senses including an audible alert, a vibrating/tapping alert, and the like. For example, the system shown in FIG. 5 shows the alerting system 508 including a haptic controller that controls a haptic motor. The haptic motor is configured to provide vibrations, taps or the like in response to the processor determining certain conditions are met, as is described later herein.

As mentioned above, the transducer may be an ultrasonic sensor 306. The ultrasonic sensor 306 is a sensor that uses ultrasonic waves which reflect off an object in a zone and such reflected signals are received by the sensor. The time of flight of the reflections are measured and the longer the time of flight is determined, the farther the object is determined to be from the transducer. The ultrasonic sensor 306 may output a numerical value to the processor based on the sensed time of flight reflections. The longer the time of flight reflections that are measured, the higher the value that is outputted by the ultrasonic sensor 306. For example, if the reflections are measured to be a time of 10 ms, the output may be a value of 10 where if the time measured is 12 ms, the output may be 12. As will be described in more depth later, this value is used by the processor to indicate (1) that there is an object in an area and (2) a relative distance measurement from the sensor that the object is located from the sensor.

It should be noted that instead of the ultrasonic sensor 306 the sensor may be any sensor or transducer that is configured for detecting that an object is within a predefined zone. As such, the present invention should not be limited to an ultrasonic sensor, which is used herein as an exemplary embodiment.

The processor 504 is configured to retrieve program modules from the memory including the module for determining alerts 510 and the module to determine object location 512. The module for determining alerts 510 includes computer instructions that are executed by the processor 504. For example, the module for determining alerts 510 includes computer instructions as to the type of alert and when an alert will be generated.

The different zones may dictate different type of alerts. For example, if the processor determines (based on the output value of the ultrasonic sensor 306) that the object is within a first zone, the alert may be a single haptic vibration/tap, but if the processor determines that the object is outside of the first zone in a second zone, the alert may be a double haptic vibration/tap (i.e., two distinct vibrations/taps).

The processor 504 also executes the module for determining alerts 510 to determine when the alerts should be issued. Alerts may be issued whenever certain predefined conditions are met, as determined by the processor. For example, the processor 504 determines if the output value from the transducer 306 is less than a predefined threshold that there is an object in a first zone.

There may be multiple predefined zones 516 (such as zones A, B, and C shown in FIG. 7) for detecting objects within the detection area of the transducer 306. The processor 504 determines, using the module to determine object location 512, if the output value from the ultrasonic sensor 306 is less than a first predefined threshold that there is an object in a zone C, is greater than a second threshold but less than the first predefined threshold that there is an object in a zone B, and is greater than a third threshold but less than the second predefined threshold that there is an object in a zone A. These predefined thresholds may be set by the manufacturer, or a user can set them using a GUI on a handheld controller device 350. The handheld controller device 350 may include a control section, which may include an operation section including an electrostatic track pad and/or a plurality of buttons that can be pressed. The operation section is disposed on the surface of the handheld controller device 350.

Referring back to FIG. 5, the processor 504 also executes the module for determining alerts 510 to determine various parameters 518 and store the parameters 518 on the system 210. For example, the alert parameters 518 may include the intensity of the alerts, the frequency of the alerts, the number of alerts, or other parameters.

Regarding the frequency of the alerts, the alerts may happen in a periodic or continuous s frequency while continuously detecting the user that the object is present to continually alert the user of the object being within the detection zone. For periodic frequencies of alerting the user, the module for determining alerts 510 may determine the frequency of the alerts should be spaced apart a predefined time period (e.g., 2 seconds). In some cases, the periodic frequency can be greater so that the user is not notified continuously, or the user can acknowledge and thus, disable the alerts by tapping the assistance system 210 or by another method.

In other embodiments, the frequency of alerts may be shorter intervals the closer the object is to the user. For example, if the object is in zone B but was in zone C (and thus, getting closer to the user), the module for determining alerts 510 may determine that the frequency of alerts be spaced closer together which will notify the user of the object getting more proximate.

In some embodiments, the alerts may have varying intensities which may signify that the object is close to the user (a greater intensity signifies that the object is close to the user (e.g., zone A) where a weak intensity signifies that the object is farther away from the user (e.g., zone C).

Thus, in light of the above, the lateral assistance system is, according to some embodiments, a spectacle mounted transducer (ultrasonic or other appropriate device) that can be mounted to the temple or other part of a conventional eyeglass frame, or attached in an alternate method to the user, that will monitor the visual field to the right or left of the user's central fixation (shown in FIG. 2) depending whether their hemianopic visual field loss is to the right or left side. The sensor will contain an alert function either within the device itself (either vibrating, tapping, audible, or some combination thereof) or connected to a haptic worn at the wrist (or other appropriate body part) via Bluetooth or other appropriate method. The alert signal when activated will notify the user that there is an object or person within the non-seeing visual field that might interfere with safe mobility. This will direct them to look into the non-seeing visual field. Controls for the range of sensitivity of the sensor and the level of haptic vibration could be user or therapist controlled by a device-mounted controller 350, by an application downloaded to a mobile or personal computing device, or other appropriate method.

The envisioned user controls might include:
1. Distance range zones: A—close (from 0 to 3 feet from the transducer); B medium (from 5-10 feet from the transducer); C far (greater than 10 feet from the transducer)
2. Frequency of range warnings: slow, medium, fast
3. Intensity of warning signal: week, medium, strong
4. Number of warning signals: one, two, three for example
5. Distance change alert sensitivity: the percent change in the obstacle distance to warrant a creation of a new alert.

In some embodiments, the system may include an object detection algorithm to detect a type of object being detected. In this regard, the system may be defined to indicate the type of object being detected and alert parameters may be defined based thereon.

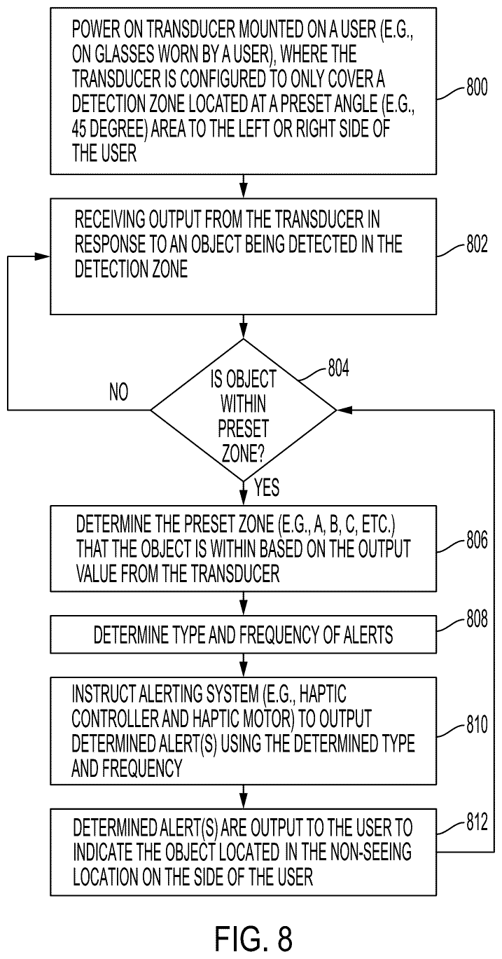

FIG. 8 is a flow diagram of an example method of the lateral awareness assistance system 210, according to some embodiments. The method may be performed by the processor 504 to detect an object and provide alerts to the user and will be described with reference to the processor 504 and transducer 306. In other embodiments, a different computing device (including, e.g., a computer/server connected via a network to the lateral awareness assistance system 210 or controller device 350) may perform the method and/or may be used to train the lateral awareness assistance system 210. To facilitate performance by a computing device (processor 504, controller device 350 or other computer/server), the method is embodied as instructions which are executable by one or more processors and stored in a non-transitory computer readable medium.

Referring to the blocks shown in FIG. 8, in block 800, the transducer 306 (e.g., ultrasonic sensor) is powered using the power source while mounted on a user (e.g., within the device, via glasses, belt, or some other method) and the transducer, according to one embodiment, is configured to only detect objected to a lateral 90 degree range to only one side of the user, as shown in FIG. 2 (either the user's left side or the user's right side). The method may use a single device that can be mounted to either the left or right temple of the eyeglass frame to detect objects on only the left side or only the right side of the user or may employ this method in each of two transducers—with a first transducer that detects objects on only the left side of the user and a second transducer that detects objects on only the right side of the user. In any event, the transducers do not occlude the user's line of sight area 102 that is directly in the line of sight of the user, as shown by reference number 102 in FIGS. 1-2.

As mentioned above, the transducer 306 outputs a numerical value when detecting an object within its detection zone. This output is transmitted from the transducer 306 to the processor 504.

In block 802, the processor 504 receives the output from the transducer 306. As explained above, if the output value from the transducer 306 is within a predetermined range or threshold, the system 210 creates an alert that indicates that an object has been located within one of the detection zones of the transducer 306.

In block 804, the lateral awareness assistance system 210 then compares the transducer output with predetermined thresholds to determine if the value is within a range of one of the predetermined zones. If so, the processor determines that the object is within the preset zones and the user should be alerted.

In block 806, if there are multiple zones set up for the lateral awareness assistance system 210, the processor then determines which preset zone (e.g., zones A, B, or C from FIG. 7) the object is within. This is done by determining which thresholds or ranges correspond to limits of the preset zones.

In block 808, the processor 504 may determine the type and frequency of alerts to be generated, as explained above. The alerts may be provided in a periodic or continuous frequency while the device is continuously detecting that the object is present within the detection zone. For periodic frequencies of alerting the user, the module for determining alerts 510 may determine the frequency of the alerts should be spaced apart a predefined time period (e.g., 2 seconds). In some cases, the periodic frequency can be greater so that the user is not bothered continuously, or the user can acknowledge and thus, disable the alerts by tapping the assistance system 210 or by another method. In other embodiments, the frequency and/or number of alerts may increase, the closer the object is to the user. For example, if the object is in zone B and was in zone C (and thus, getting closer to the user), the module for determining alerts 510 may determine that the frequency of alerts be spaced closer together which will notify the user of the object getting closer.

In some embodiments, when an object has moved into the zone, the system may require that the alert is not generated in the determined frequency (e.g., more than once) unless the output from the transducer is a certain amount greater than the threshold limit. For example, if the threshold limits for a zone is 10 to 20 and the requirement for repeated frequency alerting is that the value be more than 2 over the threshold, when the value is 11, the alert may be first provided but if the value remains at 11, the frequency (or another alert) may not continue until the value reaches 12 or the value 11 stays for more than a predetermined time threshold (e.g., 5 seconds, 10, seconds, etc.). This is because the object may go between zones and continually hit 9 and 10 repeatedly (when entering and exiting the zone) causing the system to continually alert the user when the object is not moving significantly. Thus, once the object is well within a zone (in this example, between values 12-18) or has stayed in the zone for a predetermined threshold, the alerts will be issued as normal (i.e., not be suppressed).

Also, as explained above, in some embodiments, the alerts may have varying intensities which may signify that the object is close to the user (a greater intensity signifies that the object is close to the user (e.g., zone A) where a weak intensity signifies that the object is farther away from the user (e.g., zone C).

Referring back to FIG. 8, in block 810, the alerting system (which may include a haptic motor and haptic controller) is instructed, by the processor 504 for example), to output determined alerts using the determined alert type and frequency. In this regard, the processor 504 may send a signal to the alert system with signals indicating the alert parameters, and in response to receiving those signals, the determined alerts are output to the user, in block 812.

The method may then revert back to block 804 to determine if the object is still in the detection zones and if so, repeat blocks 806-810.

If the determination in block 804 is that the object is not within the preset or predefined zones, the method will continue receiving the transducer outputs in 802 and continue looping between blocks 802 and 804 (until a positive result forces the method to block 806).

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a non-transitory computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the non-transitory computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a non-transitory computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a non-transitory computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and block diagrams of methods, apparatuses (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems which perform the specified functions or acts, or combinations of special purpose hardware and computer instructions.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “has,” “have,” “having,” “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The explicit description of the present invention has been presented for purposes of illustration and description, but is not intended to be exhaustive or limited to embodiments of the invention in the form explicitly disclosed. Many modifications and variations will be apparent to those of ordinary skill in the art without departing from the scope and spirit of embodiments of the invention. The embodiment was chosen and described in order to best explain the principles of embodiments of the invention and the practical application, and to enable others of ordinary skill in the art to understand embodiments of the invention for various embodiments with various modifications as are suited to the particular use contemplated.

Although specific embodiments have been illustrated and described herein, those of ordinary skill in the art appreciate that any arrangement which is calculated to achieve the same purpose may be substituted for the specific embodiments shown and that embodiments of the invention have other applications in other environments. This application is intended to cover any adaptations or variations of the present invention. The following claims are in no way intended to limit the scope of embodiments of the invention to the specific embodiments described herein.

What is claimed is:

1. A lateral awareness system configured to be worn by a user, the lateral awareness system comprising:
a transducer configured to only monitor whether objects are in a lateral side of a user measured from the user's line of sight;
a power source to power the transducer;
a processor to receive and process signals from the transducer to determine when alerts should be generated; and
an alerting system configured to generate the alerts.

2. The lateral awareness system of claim 1, further comprising a fastening system configured to attach the transducer to an eyeglass frame to support mobility of an individual with a visual field loss to at least one side.

3. The lateral awareness system of claim 1, wherein the transducer is configured to provide a range of outputs which vary depending on a distance of an object from the transducer.

4. The lateral awareness system of claim 3, wherein in response to the processor determining an output of the transducer is greater than a threshold indicating an object is present in a first zone, the alerting system outputs a first output.

5. The lateral awareness system of claim 4, wherein in response to the processor determining an output of the transducer is less than the threshold indicating an object is present in a second zone different from the first zone, the alerting system outputs a second output different from the first output.

6. The lateral awareness system of claim 1, wherein the transducer is attached to one or both sides of an eyeglass frame.

7. The lateral awareness system of claim 1, wherein the alert system comprises a haptic that is configured to output a vibration alert that notifies the wearer of a potential obstacle within their non-seeing visual field.

8. The lateral awareness system of claim 1, wherein transducer comprises a haptic alert that provides various vibration signals depending upon a proximity of an obstacle to the transducer.

9. The lateral awareness system of claim 1, wherein transducer that contains an optional notification light that notifies the trainer/therapist that the device has been activated to aid in the training of the user to effectively use the lateral awareness system.

10. The lateral awareness system of claim 1, wherein the transducer is directed to respond to obstacles at varying distances in the x, y and z axes of the line of sight of the user.

11. The lateral awareness system of claim 2, wherein the fastening system comprises an eyeglass mounting apparatus that can allow the lateral awareness system to be angled to control a direction of a field of view of the lateral awareness system.

12. The lateral awareness system of claim 2, wherein the fastening system comprises a central magnet combined with an array of male and female teeth for angular positioning of the device.

13. The lateral awareness system of claim 1, further comprising a circuit board that allows the transducer to be mounted at a chosen angle relative to a vertical plane representing a line of sight.

14. A method of operating a lateral awareness system, the method comprising:
receiving an output from a transducer in response to an object entering a first zone, the output of the transducer being based on a distance of the object from the transducer;
determining, by a processor, that the object is within a detection zone based on the output received from the transducer;
outputting an alert by an alerting system based on the output from the transducer.

15. The method of claim 14, wherein the user is provided with both a visual and haptic signal that increases in frequency as objects get closer to the transducer, thereby warning the user with an increased imperative for evasive action.

16. The method of claim 14, wherein the system does not continue to provide alerts for the same detected object within a set time frame.

17. The method of claim 14, wherein the system does not continue to provide warning for the same detected object within a set distance range.

18. A magnetic angle adjusting mounting system that can be clipped onto glasses frame temples for quick attachment/detachment.

19. The magnetic angle adjusting mounting system including O-rings configured for adaptability to varying frame temple profiles.

20. The magnetic angle adjusting mounting system wherein the design of the housing provides a sensor position that is not parallel to a line of sight of the user so that a sensing field is directed from a central line of sight laterally and vertically into a non-seeing lateral field of view.
